(19) United States
(12) Patent Application Publication
(10) Pub. No.: US 2009/0043208 A1
(43) Pub. Date: Feb. 12, 2009

(54) METHODS AND DEVICES FOR ESTIMATING BLOOD FLOW CHARACTERISTICS

(75) Inventors: Torbjorn Hergum, Ranheim (NO); Hans Garman Torp, Trondheim (NO)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Norwegian University of Science and Technology, Trondheim (NO)

(21) Appl. No.: 11/836,919

(22) Filed: Aug. 10, 2007

(51) Int. Cl. A61B 8/06

(52) U.S. Cl. 600/455

(57) ABSTRACT

Methods and devices are described for estimating blood flow characteristics through an orifice of a subject, such as regurgitant blood flow through a faulty heart valve. Acoustical techniques can be applied to send bursts of energy, such as high repetition pulsed ultrasonic signals, to a sample volume in a region of interest. For example, multiple beams can be formed from the bursts of energy each having a cross sectional area that is smaller than the cross sectional area of the orifice being investigated. By combining the multiple beams, a composite measure of the blood flow characteristics through the orifice can be obtained. In one example, the composite measure can provide an estimate of the cross sectional area of the interrogated orifice. The composite measure can also provide an estimate of the geometry of the orifice. Systems and components for providing such composite measures are also disclosed.

### FIELD OF THE INVENTION

The present invention is directed toward devices and methods for estimating blood flow characteristics, and more particularly to estimating such characteristics using pulse-echo signals.

### BACKGROUND OF THE INVENTION

A common condition in acquired and congenital heart disease is valvular regurgitation. That is the pathological backflow of blood through a “closed” one-way heart valve. The condition is typically manifest by the leaflets of a heart valve not closing sufficiently, resulting in a regurgitant orifice that allows reverse blood flow. Such a condition is serious, and potentially life threatening.

Understanding the characteristics of such regurgitant blood flow is important to the diagnosis and therapy of such a condition. Regurgitant blood flow can be characterized by parameters such as the rate of blood flow through the defective valve and the cross sectional area allowing regurgitant flow when the leaflets are “closed”. The use of noninvasive techniques for diagnosing the condition can be beneficial. One existing technique is the use of ultrasonic real-time imaging using Doppler signals. An ultrasonic pulsed Doppler signal is acquired from a region of interest completely enveloping the regurgitant valve. The signal is used to form a power-velocity spectrum. To obtain absolute measurements of area and velocity, a reference Doppler signal from a region of interest within the regurgitant flow is acquired. Knowing the characteristics of this reference signal allows calibration of the actual measurement signal. From these measurements, estimates of the cross-sectional area of regurgitant blood flow can be obtained.

The aforementioned method, however, can provide poor estimates. For example, use of a single ultrasonic pulsed Doppler signal to ensonify the entire sample volume assumes that the measurement has uniform spatial sensitivity over the entire cross sectional area of a regurgitant blood flow jet to be examined. This, however, is often not the case as the spatial sensitivity can vary across the cross-sectional area to be interrogated, leading to a poor estimate of the blood flow characteristics. As well, the use of a single beam does not allow the geometry of the orifice to be determined. Furthermore, these estimates of cross-sectional area depend upon spectral analysis of the received Doppler signal, which complicates the process of obtaining the estimate.

Accordingly, a need exists for improved devices and methods for characterizing the blood flow from a regurgitant valve, and to provide a more accurate depiction of regurgitant blood flow in a subject for diagnosis and therapy.

### SUMMARY OF THE INVENTION

One exemplary embodiment is directed to a method for estimating characteristics of blood flow through an orifice of a blood vessel. Such an orifice can be a dynamic orifice, such as a heart valve. Multiple pulse-echo signals (e.g., ultrasonic Doppler signals) from a region of interest, which can envelope an opening area of the orifice, can be acquired. An example of a region of interest can be the vena contracta region of jet blood flow (e.g., regurgitant blood flow) through the orifice. The multiple pulse-echo signals can be generated as a result of a high pulse repetition frequency technique. One or more of the pulse-echo signals can have a sample area intersecting the opening area of the blood vessel orifice (e.g., the pulse-echo signal can have a sample area located within the opening area). As well, the multiple pulse-echo signals can be filtered to suppress signal contributions from slow moving scatterers (e.g., scatterers moving below a threshold velocity). Subsequently, the multiple pulse-echo signals can be assembled to form a composite measure, which can provide an estimate of blood flow characteristics through the orifice. In one instance, the composite measure can be a composite power distribution, with multiple power signals optionally generated. Each power signal can correspond with one or more of the multiple pulse-echo signals. In another instance, the composite measure can provide an estimate of the geometry of the orifice.

In a related aspect of the exemplary embodiment, a cross sectional area of blood flow through the orifice can be estimated. Such an estimate can be obtained without the use of spectral analysis conducted on the pulse-echo signals. In one instance, the cross sectional area can be estimated using the measured power of multiple pulse-echo signals. In this instance the pulse-echo beam profile corresponding with each of the pulse-echo signals can be used. For example, a pulse-echo beam profile for each of the pulse-echo signals can be estimated. The pulse-echo beam profiles can then be assembled to form a composite beam profile. The composite beam profile can be used to estimate the cross sectional area of blood flow through the orifice. A reference pulse-echo signal can be identified from the multiple pulse-echo signals. The reference pulse-echo signal can correspond with the pulse-echo signal having the highest power. A corresponding reference signal power can be identified. Also, a corresponding reference beam profile, which can be one of the estimated multiple beam profiles, can be identified. The cross sectional area of blood flow can then be calculated from the estimated total power of the pulse-echo signals, the reference signal power, the reference beam profile, and the composite beam profile.

Using any of the estimates for the cross sectional area discussed herein, an average value of blood flow through the orifice can be obtained. The multiple pulse-echo signals can be used to estimate the average velocity of blood flow through the orifice. For example, a velocity can be associated with each of the multiple pulse-echo signals, and those individual velocities can be combined to form the average velocity. The average velocity can be combined with the cross sectional area to estimate the blood flow.

These techniques for estimating the cross sectional area of blood flow can also include obtaining multiple sets of power signals from corresponding sets of pulse-echo signals. Each set of pulse-echo signals can be obtained over a unique time interval. The reference signal power can be identified by combining (e.g., averaging) multiple received beam power values. Each of the received beam power values can correspond with one of the multiple pulse-echo signals that have a cross sectional area intersecting (e.g., located within) the orifice's opening area. The reference signal power can also be provided by identifying spatially corresponding reference pulse-echo signals taken at different time intervals. Power values corresponding with each of the reference pulse-echo signals can be combined to provide the reference signal power. An estimated total power can be provided by estimating a set-total power value from a corresponding set of power signals, and calculating the total power by combining the multiple set-total power values.

Another exemplary embodiment is directed to an acoustical system for interrogating blood flow through an orifice of a blood vessel. The system can be configured to produce multiple pulse-echo signals (e.g., Doppler echo signals) and can optionally be configured to produce color flow images of the blood flow. The system can include a pulse generator, which can be coupled to one or more transducers, for transmitting bursts of energy (e.g., ultrasonic energy), which can be directed toward a region of interest, such as an opening area of the orifice. The bursts can be delivered in a high pulse repetition frequency mode. One or more transducers can be included for receiving multiple echo signals. Each echo signal can correspond with a backscattered signal from one or more of the transmitted bursts of energy. The system can optionally perform beamforming on the transmitted signal and/or the received signals to have a sample volume with a cross-sectional area smaller than the opening area of the orifice. The transducer(s) that are coupled to a pulse generator for producing the bursts of energy can be the same transducer(s) as those configured to receive the echo signals. Alternatively, separate transducers can be used to supply the energy and capture backscattered signals. A signal processor can be coupled to one or more transducers. The signal processor can be configured to convert the multiple echo signals into a composite blood flow measure such as a spatial power distribution. To remove portions of signals that correspond to slow moving scatterers or other unwanted artifacts, a filter (e.g., a high-pass filter) can optionally be included, which can be coupled to a transducer and the signal processor.

In some embodiments, the pulse generator and the transmitting transducer(s) can be configured to produce multiple sets of transmitted energy bursts, which can be arranged such that each set of bursts is produced during a unique time period. In such embodiments, the signal processor can be configured to calculate a combined-echo-signal power from the multiple sets of transmitted energy bursts. In particular, each set of energy bursts can correspond to a set of echo signals. Each set of echo signals can each be converted to a corresponding set-total power value, and the individual set-total power values can be combined to form the combined echo-signal power.

Other embodiments configure the signal processor of the acoustical system to associate a beam profile with each echo signal. A composite beam profile can be estimated from a combination of the individual beam profiles. This composite beam profile can be used by the signal processor to estimate a cross sectional area of blood jet flow emitted from the orifice; such an estimate can also be provided without the processor needing to conduct spectral analysis on the echo signals (e.g., without individually analyzing separate frequency components). This estimate can also depend upon a composite power value, which can be derived from the composite spatial power distribution. The estimate of cross sectional area of blood flow from the orifice can also be provided by the signal processor by configuring the processor to choose a reference echo signal from the multiple echo signals (e.g., the signal with maximum power of an average, or all pulse-echo signals with sample area intersecting with the opening area of the orifice). The reference pulse-echo signal can correspond with an echo signal from at least one of the pulsed energy bursts which interacts with scatterers in a sample volume intersecting with the opening area of the orifice.

### BRIEF DESCRIPTION OF THE DRAWINGS

The invention will be more fully understood from the following detailed description taken in conjunction with the accompanying drawings (not necessarily to scale), in which:

FIG. 1 presents a schematic side view of a transducer used to ensonify regurgitant flow through a heart valve, in accord with an embodiment of the invention;

FIG. 2 presents a schematic cross sectional view of a leaky blood vessel valve being interrogated by a plurality of beams;

FIG. 3 presents a graph of power versus position a pulse-echo beam profile from a rectangular transducer;

FIG. 4 presents a graph of multiple individual pulse-echo beam profiles modeled as sinc-shaped beams and the composite pulse-echo beam profile created by summing the individual beam profiles;

FIG. 5 presents a schematic flow diagram of a system for providing a composite measure of the blood flow characteristics through an orifice, in accord with an embodiment of the invention;

FIG. 6 presents a graph of the estimated area versus the programmed area for a simulation utilizing embodiments of the present invention;

FIG. 7 presents a graph of the estimated area versus the programmed area for a stochastic Doppler-model simulation in accord with an embodiment of the invention;

FIG. 8A presents graphs depicting the geometry of a modeled orifice and the composite beam profile g_{w}(x,y,τ) for a simulation; and

FIG. 8B presents a graph of the power distribution associated with each of the individual beam profiles used to form the composite beam profile g_{w}(x,y,τ) depicted in FIG. 8A.

### DETAILED DESCRIPTION OF THE INVENTION

Certain exemplary embodiments will now be described to provide an overall understanding of the principles, structure, function, manufacture, and/or use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with features of other embodiments. Such modifications and variations are intended to be included within the scope of the present invention.

### Methods of Interrogating Blood Flow Characteristics

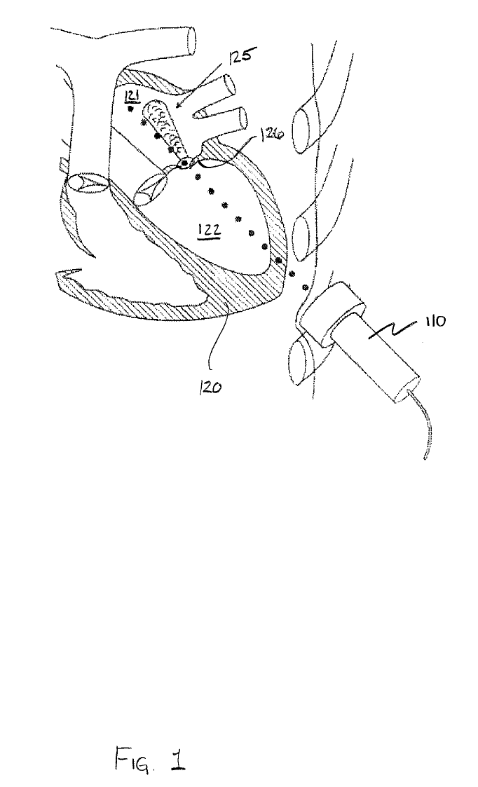

FIG. 1 provides a schematic of an exemplary situation in which some embodiments of the invention can be utilized. The heart 120 of a human subject is depicted, with blood flow typically moving from the left atrium 121 to the left ventricle 122 through an atrioventricular valve 126. As depicted, the heart 120 has a faulty valve 126, in which the leaflets of the valve 126 do not meet when the valve closes. As a result, a jet of blood 125 flows from the left ventricle 122 back through the valve 126 and into the left atrium 121, though the valve 126 should close off blood flow. To assess the characteristics of this faulty blood flow in this particular depiction, a transducer 110 is placed proximate to a patient to send pulses of ultrasonic energy toward a region of interest near the faulty blood flow region. These pulses of ultrasonic energy interact with the scatterers in the blood (e.g., red blood cells), resulting in corresponding backscattered echo signals, also known as a pulse-echo signals or echo signals. The phrase “pulse-echo signal” as utilized herein refers to the echo signal corresponding to a specific burst of energy. A pulse echo signal can, optionally, include beamforming performed on transmit and/or receive, and other signal processing functions such as those discussed herein. In general, assuming a constant hematocrit level for the blood, and that red blood cells can be approximated as delta-correlated, the power of the backscattered echo signal is proportional to the volume of blood interrogated by the energy burst resulting in the echo signal.

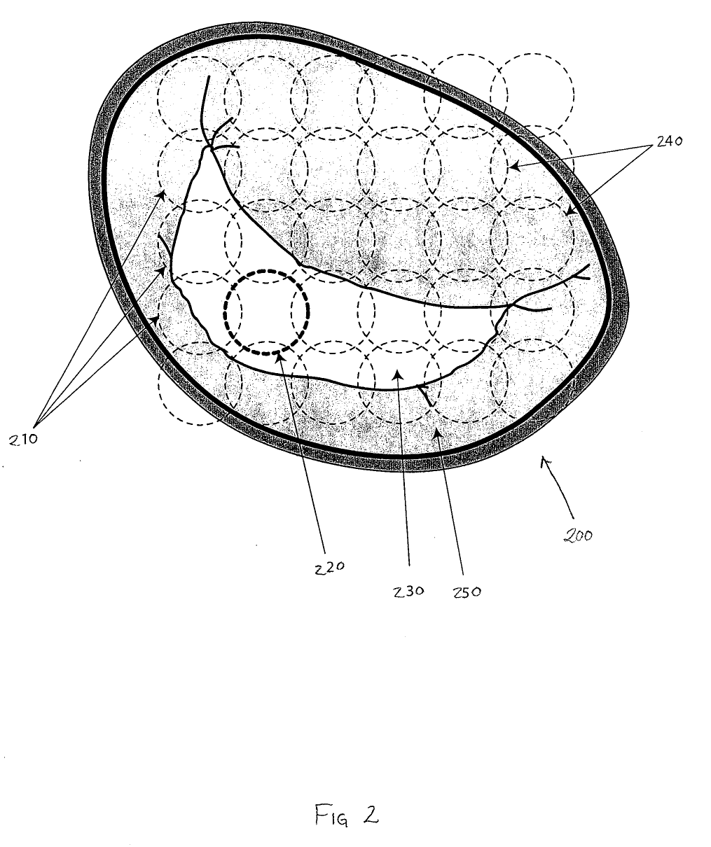

To examine the portion of a signal corresponding to a particular sample volume, which was ensonified by an energy burst, a return signal can be captured over a designated time period. By only examining the signal over the designated time period, signals produced by a corresponding thickness region are interrogated. As well, the corresponding cross sectional area of the sample volume can be controlled by the geometry of the transducer(s) used to capture the echo signal, among other techniques discussed in more detail herein. Accordingly, to measure the regurgitant blood flow, a sample volume can be chosen to be a thin disc-like shape positioned directly adjacent to the regurgitant side of the valve 126 at the vena contracta for the situation depicted in FIG. 1. When multiple echo signals are utilized, the signals can be received and processed to form a composite measure of the characteristics of blood flow through the valve 126. For example, each of the multiple pulse-echo signals can correspond with a different interrogated sample volume, where the volumes can optionally overlap to some degree as depicted in FIG. 2. Each sample volume interrogated can result in an individual measure of the characteristics of blood flow in that sample volume. Thus, the composite measure can be a combined measure over some entire representative volume interrogated by the multiple echo signals.

The phrase “composite measure” as utilized herein refers to one or more quantities used to characterize a collective trait such as blood flow characteristics. For example, the quantities can be a single value such as a power, velocity, cross sectional area, and/or other measure of flow characteristics through a leaky valve. The quantities can also be a distribution of values over a spatial region, such as a power distribution or velocity distribution over the opening area of an orifice. The term “measure” as utilized herein refers to one or more quantities related to some determination of extent in one or more delineated parameters. Accordingly, a “measure” is not necessarily in the form of an absolute value of a delineated parameter. For example, a measurement of power need not be in typical power units but can be in arbitrary units that allow a comparison between different measures of power without identifying an absolute quantity (e.g., the power can be normalized on some physical, or arbitrary, scale). Of course, a measure can be an extensive variable, though without any limitation to using a particular system of units (e.g., CGS, MKS, English, etc.). Furthermore, measures can be represented by any value that can be converted to the desired delineated parameter. For example, a power value can be correlated with the square of a received signal value, and can also be correlated with the volume of scatterers that a pulse interacts with. As such, those skilled in the art will appreciate that a “measure” can be represented in various manners that can be utilized within the scope of embodiments of the present application.

Accordingly, some embodiments of the invention are drawn to methods and devices for estimating blood flow characteristics through an orifice of a subject's blood vessel. In some instances, embodiments can be used to determine the characteristics of blood flow through a dynamic orifice, such as a valve of an organ (e.g., the human heart). Thus, some embodiments can provide blood flow characteristics of a faulty heart valve in which regurgitant blood flow can occur. In particular, a plurality of pulse-echo signals from a region of interest, e.g., the vena contracta of a blood flow jet, can be assembled to form a composite measure of the blood flow characteristics through the orifice. The composite measure can take a number of forms, such as a power distribution, a velocity distribution, a measure of the cross-sectional area of blood flow (e.g., through a jet of an orifice), or a measure of the geometry of the orifice. These composite measures are one novel aspect of the present application.

In some embodiments, the plurality of pulse-echo signals are configured to cover a region of interest, which can envelop an opening area to be interrogated. The opening area can be, for example, any cross-section through which fluid can flow, and be interrogated. In many embodiments herein, the opening area corresponds with a cross-sectional area of blood flow, e.g., an area adjacent to the outlet of regurgitant jet blood flow 125, such as the vena contracta, as depicted in FIG. 1. It is understood that the methods and devices disclosed, herein, can be used to interrogate any number of types of fluid flow, and are not restricted necessarily to the particular applications explicitly discussed herein.

FIG. 2 provides a cross-sectional view of a faulty valve 200 being interrogated by multiple energy bursts. In particular, when the leaflets 250 of the valve 200 are closed, an orifice 230 remains that allows the back flow of blood through the valve 200. The multiple energy bursts can be focused and steered to interrogate the region of interest. For example, the region of interest can be interrogated by examining a set of thin-sectioned volumes (e.g., disk-shaped). These volumes can be partially overlapping, and can be used to interrogate a larger volume, such as a thin slice, in the vicinity of the orifice 230. Such volumes can each have a cross-sectional area 210 defined to individually intersect the cross-sectional area of the orifice 230 (e.g., one or more of the cross sectional areas 220 can be located within the cross sectional area of the orifice 230).

With respect to FIG. 2 and as discussed above, each of the thin-sectioned sample volumes, corresponding to thin disk-like volumes having a cross sectional area 210, can be defined by a portion of a corresponding echo signal that is examined. The thickness of the disk-shaped sample volumes can be defined by the time interval of the echo signal that is captured and observed, as discussed above. The cross-sectional area 210 of each sample volume can be defined by the characteristics and positioning of one or more transducers that are used to transmit the energy burst and/or receive the echo signal. For example, modifying the aperture of a transducer used to transmit an energy burst can result in a narrower or wider cross-sectional area being interrogated. This modification of the transducer aperture can be performed by physically using a smaller or larger transducer, and/or electronically applying independent weightings to each transducer element used for transmitting and/or receiving. The latter approach is a technique known as “apodization.” Further details regarding controlling the cross-sectional area, i.e., lateral dimensions, interrogated can be obtained in U.S. Pat. No. 6,544,181 B1 of Buck et al., issued Apr. 8, 2003. The teachings of the patent are hereby incorporated herein by reference in their entirety.

As used throughout the present application, the term “beam” refers to an ultrasound pattern associated with a particular echo signal. The pattern can be influenced by the focusing and/or steering of bursts of energy produced by a transducer towards a desired spatial location, which subsequently affects the associated received echo signals. The pattern can also be influenced by a receiving transducer, which receives an echo signal and applies steering and/or focusing thereto. The term “sample volume” is used to refer to the echoes corresponding to a specific depth. The cross sectional area of the sample volume is given by the cross sectional area of the beam. The length of the sample volume is determined by the pulse length and the range gate of the receiving transducer.

In some embodiments, a plurality of beams have a cross sectional area 210 smaller than the cross sectional area of the interrogated orifice 230, as exemplified by the depiction in FIG. 2. Using beams with this size cross sectional area can enhance interrogation of the orifice relative to earlier methods that utilize wider beams, i.e., sample volumes with larger cross sectional areas. For example, using a number of beams with smaller cross sectional areas can allow accounting for the varying spatial sensitivity of a wide beam across an orifice area. As utilized herein, the “beam profile” of a transmitting transducer refers to the spatial distribution of energy at a given depth. For a receiving transducer, the term “beam profile” refers to the spatial sensitivity for returning echoes from a specific depth. The term “pulse-echo beam profile” refers to the combination of the transmit beam profile and the receive beam profile, which provides a total spatial sensitivity of the pulse-echo system at a specific depth. Accordingly, a flat pulse-echo beam profile refers to a configuration of the pulse-echo system where the spatial sensitivity is uniform at a given depth over the area of interest, while a non-uniform pulse-echo beam profile can refer to a configuration of the system in which the sensitivity varies substantially as a function of spatial position.

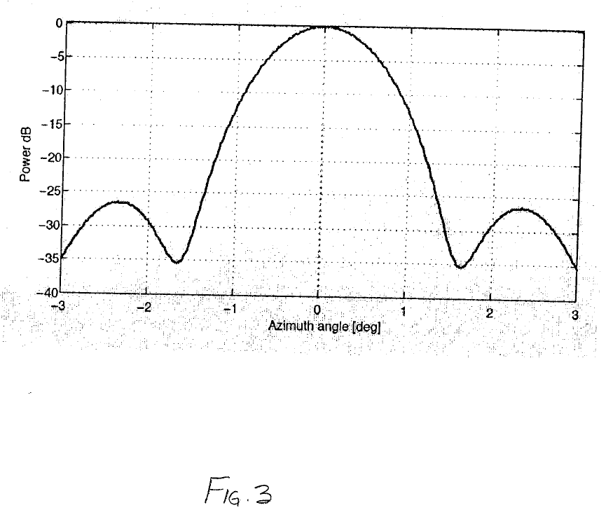

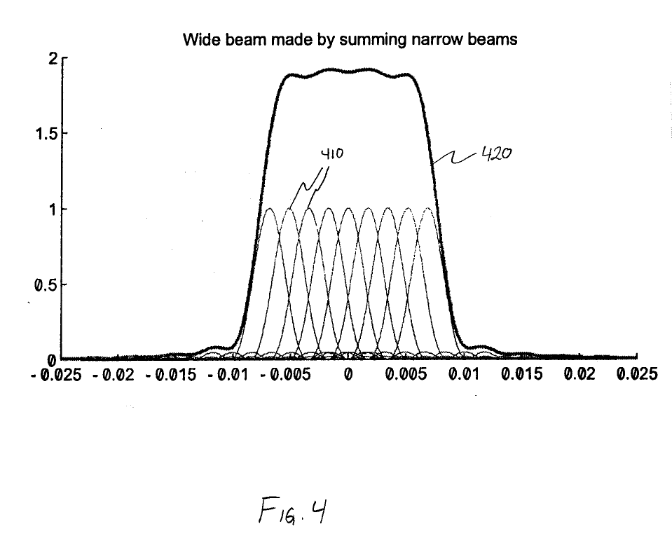

FIGS. 3 and 4 provide graphs of exemplary beam profiles. FIG. 3 depicts an exemplary 2D cross section of a 3D pulse-echo beam profile from a rectangular transducer. As the profile is not flat, the received signal power from scatterers located off-center from the beam will be different from on-axis scatterers. This effect can lead to poor composite measure calculations of blood flow characteristics, such as a cross-sectional area of the orifice. In contrast, using a plurality of smaller cross sectional beams can allow for interrogation of an entire cross-sectional area of an orifice with less error due to backscatter signal sensitivity. For example, an overall pulse-echo signal can be estimated using 9 narrow pulse-echo signals that are overlapping. This is presented in FIG. 4, where the beam profiles from 9 overlapping, narrow beams 410 are summed to provide an overall pulse echo beam profile 420 that is much flatter than a single beam of similar width. In this example, the narrow beam profiles are estimated using sinc functions and they are spaced at 0.5 times the Rayleigh criterion. Using less dense spacing will make the ripple in the overall profile 420 more prominent. Accordingly, some embodiments utilize multiple beams that are substantially evenly distributed over the orifice cross sectional area with at least some of the beams having a cross sectional area within the orifice cross sectional area. In any event, by obtaining characteristics of each of the smaller cross sectional area beams, distributions of power and/or velocity, as well as a detection of the geometry of the orifice, can be obtained as described further herein.

The Nyquist frequency describes the minimum sampling frequency needed to be able to resolve the blood velocity without aliasing. Specifically, the pulse repetition frequency (PRF) must be at least double the maximal Doppler shift frequency in the pulse-echo signal to prevent aliasing. The lower bound of the PRF is generally given by the aliasing limit, and the higher bound of the PRF is given by the time it takes from one pulse being fired until the corresponding echoes have died down enough not to interfere significantly with the echoes from the next pulse. If the PRF is increased above the higher bound to be able to resolve higher velocities without aliasing, range ambiguity occurs. This is called a high pulse repetition frequency (herein “HPRF”) technique. The pulse-echo signals used in embodiments of the present invention can be generated using any number of methods, though it can be advantageous to employ a HPRF technique.

In some embodiments, the plurality of pulse-echo signals can be filtered before being assembled to form one or more composite measures of blood flow characteristics. Embodiments can optionally utilize a clutter-filter, sometimes called a fixed target canceling (FTC) filter, on the pulse-echo signal. The filter can eliminate signals from slow-moving blood and tissue in the sample volume and/or in the HPRF-induced ambiguous sample volumes. The types of filters that can be utilized include all those appropriate for removing clutter or low frequency noise, including filters known to those skilled in the art.

Pulse-echo signals obtained in different subjects, or at different moments of time, can vary due to changes in attenuation and backscattering coefficients. To be able to provide an absolute measure of the blood flow characteristics a calibrated measurement can be performed in each subject. In some embodiments, a reference beam can be utilized to help obtain one or more composite measures of blood flow characteristics. The reference beam can serve to help calibrate a signal measurement to provide absolute quantities of a desired characteristic.

In some embodiments, the reference beam is chosen to correspond with one of the multiple pulse-echo signals acquired and assembled to form the composite measure, as opposed to being a completely separate signal as utilized in earlier techniques. The reference beam can correspond with one of the pulse-echo signals having a cross sectional area within the cross sectional area of the orifice being measured, e.g., the pulse-echo signal having the maximum power relative to all signals within the cross sectional area of the orifice. In other embodiments, the reference beam can correspond with multiple signals that are combined in some fashion to provide the reference beam. In one instance, multiple pulse-echo signals are obtained from corresponding spatial positions (e.g., substantially the same position relative to an orifice position) at different moments in time. The signals are combined in some fashion, for example calculating a power measure associated with each signal, and averaging the power measures together. As used in the present application, the terms “average” and “averaging” refer to combining multiple measures or values to obtain some composite measure or value, which is representative of the individual measures and values. The use of the terms “average” and “averaging” are meant to be broadly interpreted to include any number of value or measure combining techniques including those understood by those skilled in the art. Types of averages include arithmetic averages, geometric averages, weighted averages using various weighting techniques (e.g., weighted relative to some metric). In another instance, multiple beams that each have a cross-sectional area that overlaps the cross sectional area of the orifice are combined. For example, power values corresponding to beams with cross sectional areas within the cross sectional area of the orifice can be averaged to produce an average power value, which is used as a reference value.

In the techniques described above for choosing/obtaining a reference beam, it can be advantageous for the technique to be implemented in an automated fashion, e.g., using a programmed microprocessor. However, the reference beam can also be selected with user-controlled techniques. For example, the reference beam can be calculated based on a selection of an area or time-slice made by a user. The selection can, for instance, be made using a pointing device on a screen displaying for instance the estimate of the geometry of the orifice. Some or all of the beams selected can serve as the basis for calculating a reference beam. Other techniques for choosing and/or obtaining a reference beam, or corresponding value or measure, can also be utilized with various embodiments disclosed herein.

### Estimating the Cross-Sectional Area of Blood Flow Through an Orifice

Some embodiments are directed to developing estimates of blood flow characteristics through an orifice such as the cross-sectional area of blood flow. One exemplary methodology of estimating the area of flow is described hereforth. This methodology can be applied without performing detailed spectral analysis on the pulse-echo signals (e.g., specific frequency components of the pulse-echo signals need not be identified, parsed, and/or analyzed to provide the estimate), thus simplifying the cross sectional area determination relative to known methods. As well, this methodology, and parts of the methodology, can be used in other embodiments described herein to provide other composite measures or values. Those skilled in the art will readily recognize other variations in this methodology that allow alternative methodologies to be utilized that are not explicitly discussed by the present disclosure. All these techniques, however, are considered to be encompassed within the scope of the present application.

Let g(x,y,τ) represent a pulse-echo beam profile, where x and y are the azimuth-axis coordinate and the elevation-axis coordinate, respectively, and τ is the time after pulse transmission. Let N represent the number of pulse-echo signals utilized, with each pulse signal centered at a location (xₖ, yₖ), k=1:N. Accordingly, the pulse-echo beam profile for an individual signal is represented by gₖ(x,y,τ).

If sₖ(τ) is the received signal from a beam steered to the position (xₖ, yₖ), where τ is the time delay after pulse transmission, the expected power of the beam is given by

$${P}_{k} ( τ ) = 〈 {\vert {s}_{k} ( τ ) \vert}^{2} 〉 = {\int }_{- ∞}^{∞} σ {\vert {g}_{k} ( x , y , τ ) \vert}^{2} d x d y \qquad ( 1 )$$

where < . . . > denotes the use of the expectation value operator and a takes into account the scattering cross section and the removal of energy due to clutter filtering.

Using a plurality of signals to provide a composite measure, the expected total received power from all the signals can be determined by the sum of expected power from the individual signals as obtained from (1),

$${P}_{w} ( τ ) = {\sum }_{k} {P}_{k} ( τ ) = 〈 {\sum }_{k} {\vert {s}_{k} ( τ ) \vert}^{2} 〉 = {\int }_{CSAjet} {\sum }_{k} σ {\vert {g}_{k} ( x , y , τ ) \vert}^{2} d x d y \qquad ( 2 )$$

where P_{w}(τ) is the expected total received power, CSAⱼₑₜ indicates that the integration is performed over the cross sectional area of the jet. It is sufficient to perform the integration over the cross-sectional area of the jet since this is the area that has scatterers that are fast enough to give a Doppler-shift above the cut-off frequency of the applied clutter-filter. Optionally, a threshold filter can be applied to remove low-power values remaining due to non-ideal clutter filter performance.

Choosing a reference beam, as discussed earlier, the expected received power of the reference beam can be represented by

$${P}_{ref} ( τ ) = 〈 {\vert {s}_{kref} ( τ ) \vert}^{2} 〉 = {\int }_{- ∞}^{∞} σ {\vert {g}_{ref} ( x , y , τ ) \vert}^{2} d x d y \qquad ( 3 )$$

where the reference beam is directed to the position (x_{kref}, y_{kref}), and the corresponding relationship from equation (1) is applied to represent the expected value of the power of the received reference signal.

Let G_{w}(x,y,τ) represent the two-way power beam profile of the composite beam, which is given by

$${G}_{w} ( x , y , τ ) = {\sum }_{k} {\vert {g}_{k} ( x , y , τ ) \vert}^{2} \qquad ( 4 )$$

Also, let Γ_{ref}(τ) be the two-way reference power beam profile, which is given by

$${Γ}_{ref} ( τ ) = {\int }_{- ∞}^{∞} {\vert {g}_{ref} ( x , y , τ ) \vert}^{2} d x d y \qquad ( 5 )$$

Dividing equation (2) by equation (3), and inserting the relationships for equations (4) and (5), the following relationship can be derived:

$$\frac{{P}_{w} ( τ )}{{P}_{ref} ( τ )} = \frac{{\int }_{CSAjet} σ {G}_{w} ( x , y , t ) d x d y}{{Γ}_{ref} ( τ )} \qquad ( 6 )$$

When a high number of beams are used to provide a composite beam that extends beyond the area of the jet, the two-way power beam profile can be considered constant over the jet area.
In such an instance, the value of G_{w}(x,y,τ) for a chosen time lag τ can be estimated as being a constant value G_{w}. Accordingly, equation (6) can be rearranged and simplified to provide the following estimated relationship for the cross-sectional area of the jet,

$${CSA}_{jet} = \frac{{P}_{w} ( τ )}{{P}_{ref} ( τ )} \frac{{Γ}_{ref} ( τ )}{{G}_{w} ( τ )} \qquad ( 7 )$$

The area estimated by equation (7) is dependent upon an angle θ between the beam and the normal of the cross-sectional area of the orifice. Accordingly, CSAⱼₑₜ as calculated by equation (7) can be multiplied by a factor of cos θ to correct for the angular dependence.

The described methodology can be applied to estimate the cross sectional area of an orifice. The beam profiles g(x,y) corresponding to each of the pulse-echo signals can be determined in a number of ways. For instance, a Fraunhofer approximation can be utilized to estimate the beam profile from the Fourier transform of the transducer aperture. As well, the beam profiles can be found from time-domain simulations as implemented in software packages such as Field II, or by conducting calibration experiments with the equipment used to provide the received signals. As an example, each beam profile can take the form of the profile shown in FIG. 3, which was calculated using the Fraunhofer approximation of the beam profile from a rectangular transducer. Indeed, all these potential methods are known to those skilled in the art, and these methods and others are all contemplated to be used with the disclosed embodiments. Thus, the individual beam profiles can be used for the calculation of the cross sectional area of blood flow through an orifice, among other embodiments disclosed in the present application.

One exemplary embodiment of estimating the cross sectional area of an orifice assumes a form for the individual beam profiles, as described above. These individual beam profiles can then be used to represent Γ_{ref} and G_{w}. P_{w} and P_{ref}, the total received power and reference power, are obtained from the corresponding received pulse-echo signals; P_{w} being the sum of squares of the received individual signals and P_{ref} corresponding to the square of the reference signal. P_{ref} can be chosen as the maximum power of the individual pulse-echo signals (e.g., within the cross sectional area of the orifice) or an average power over time or over some or all the beams in the cross sectional area of the orifice. The cross sectional area can be obtained using equation (7), inserting P_{w} and P_{ref}. The values for Γ_{ref} and G_{w} can be obtained using equations (5) and (4), respectively.

In one variation of the exemplary embodiment, multiple sets of pulse-echo signals are obtained and combined (e.g., averaged) to provide the estimate of the cross-sectional area of the orifice with blood flow. In one instance, each set of pulse-echo signals includes signals that correspond with scattering during a unique time interval. For example, only pulse-echo signals corresponding to a particular set undergo scattering in the region of interest during a selected time interval, all other pulse-echo signals undergo scattering in the region of interest substantially outside the selected time interval. Accordingly, a set-total power value can be estimated for each of the sets of pulse-echo signals (e.g., by summing the power values associated with each of the pulse-echo signals in a set). These set-total power values can be combined (e.g., averaged) to provide a total power, which can represent an effectively time-averaged power value.

In addition or alternatively, a reference signal can be corresponded for each of the sets of pulse-echo signals. The reference signals can be chosen so that they all correspond spatially to approximately the same location relative to a position of the orifice. The reference signals can be converted to corresponding received beam power values, which can be combined (e.g., averaged) to form a combined reference power value. This combined reference value can be used with the total power value derived above to estimate a cross sectional area for blood flow through the orifice. Of course, other variations of this exemplary embodiment can also be practiced. For example, the total power can be calculated from sets of pulse-echo signals, though only a subset of measurements of a reference signal (e.g., only one reference signal) is utilized. As well, the total power from a subset of pulse-echo signals is calculated (e.g., from only one set), while an average reference signal is calculated using a signal from each of the sets of pulse-echo signals.

Other embodiments can utilize the cross sectional area of the orifice to obtain other blood flow characteristics. For example, the average blood flow rate through the orifice (e.g., the average flow rate of the vena contracta) can be found by multiplying the estimated cross sectional area by the average velocity of the blood flow. Alternatively, individual velocity values can be associated with each of the pulse-echo signals. By multiplying a pulse-echo signal cross section by the corresponding individual velocity and summing the products, an estimate of the average blood flow rate can be obtained. Methods of obtaining the corresponding velocity measurements are described herein, and include methods well understood by those skilled in the art.

### Signal Distribution Values and Orifice Geometry

The description above provides techniques for determining the cross sectional area of blood jet flow from an orifice. Some of the embodiments involved combining multiple individual pulse-echo signals in a variety of fashions to obtain the cross sectional area of interest. The individual pulse-echo signals, however, can also be used to provide characteristic values for each of the individual pulse-echo signals. The distribution of these characteristic values, i.e., the values and the geometric relation of the corresponding pulse-echo signal relative to the measured orifice, can also provide a measure of the geometry of the orifice. Accordingly, in some embodiments of the invention, a composite measure from multiple pulse-echo signals can be embodied as a distribution of values that provide an estimate of the geometry of the blood flow jet orifice. In one example, the individual power values of each pulse-echo signal (e.g., the square value of the received signal) can be compared to one another using a graphical representation, or through some value examination technique. For power values that fall below a threshold value (e.g., less than 12 dB of the maximum power value for an individual pulse-echo signal), the corresponding pulse-echo signal can be considered an edge of the blood flow jet. Accordingly, a measure of the geometry of the cross sectional area of the orifice or blood flow jet is provided. It is understood that though absolute power values can be utilized in some embodiments, others need not utilize an absolute power value, e.g., the variance or the Doppler shift of the signal can be used.

Of course, a variety of such values can be calculated and associated with each pulse-echo signal to provide a measure of geometry. Non-limiting examples include power, velocity, variance, or other values indicative of blood flow (which can be instantaneous, time-integrated, time-averaged, etc.). In particular, Doppler velocimetry is a technique well understood by those skilled in the art, which can be utilized with various embodiments to provide a velocity distribution of flow through an orifice. In an exemplary embodiment, an individual received pulse-echo signal (e.g., corresponding to a beam with a cross-sectional area within the cross sectional area of the orifice) can be analyzed with respect to its Doppler shifted center frequency. The Doppler frequency can be related to the velocity of the corresponding scatterer using the relationship

$${f}_{D} = 2 \frac{f}{c} {V}_{SC} cos θ$$

where f_{D} is the particular received Doppler frequency, f is the frequency of the pulse signal sent, c is the velocity of sound in the medium, V_{SC} is the velocity of the scatterer, and θ is the angle between the direction of scatterer and the pulse-echo signal direction. A further description of some of these techniques is provided in U.S. Pat. No. 6,544,181 B1 of Buck et al., issued Apr. 8, 2003.

Other measures of velocity can be associated with an individual pulse-echo signal without detailed analysis of a large number of frequency components (e.g., using the mean frequency component to associate a velocity value). As noted above, beyond using velocities associated with individual pulse-echo signals, the velocities can be combined or obtained in other manners to calculate overall composite measures such as an average blood flow rate through a regurgitant orifice.

### Systems for Interrogating Blood Flow Through a Blood Vessel Orifice

Some embodiments of the invention are directed to systems for interrogating blood flow characteristics through a patient using an acoustical (e.g., ultrasound) technique. Though these embodiments can be configured to practice one or more steps of the methodologies discussed in the present application, it is understood that the system is not necessarily limited to the functionalities explicitly described herein. Some embodiments can include a pulse generator, which can be configured to transmit bursts of energy (e.g., ultrasonic energy) toward an orifice to be interrogated. One or more transducers can be included. These transducers can receive multiple echo signals (e.g., Doppler signals), where each echo signal can correspond to a backscattered signal from one or more of the transmitted energy bursts. A signal processor can be coupled to the transducer(s). The processor can convert the echo signals to a composite measure, such as a composite spatial power distribution.

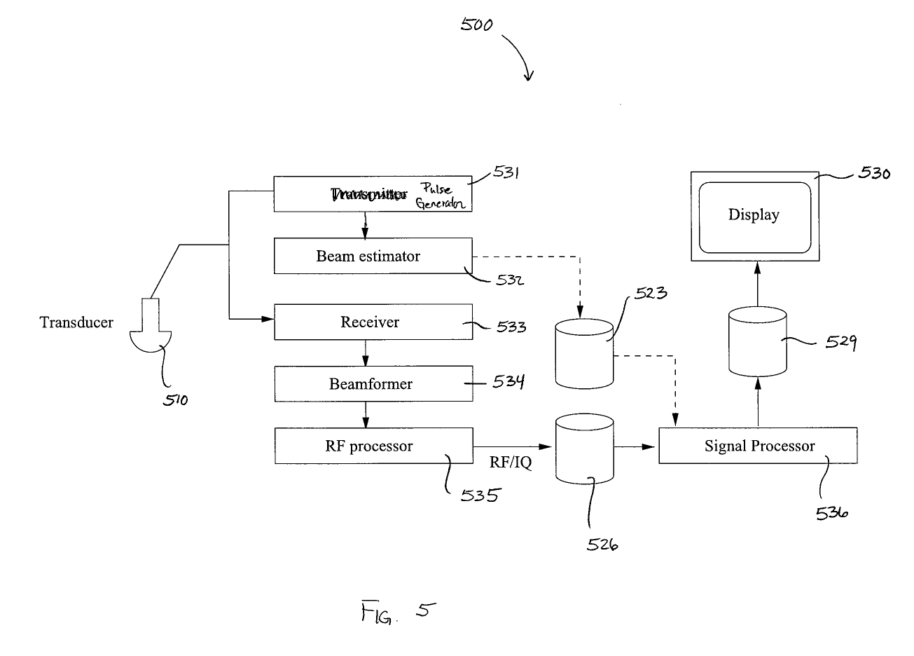

FIG. 5 provides an exemplary system for interrogating blood flow through an orifice, such as one of the valves of a heart in which regurgitant blood flow occurs, consistent with some embodiments. For the system 500 shown, a transducer 510 is coupled to a pulse generator 531 and receiver 533. A beam estimator 532, beamformer 534, and RF processor 535 can be used in conjunction with a signal processor 536 to form a composite measure of blood flow characteristics through the orifice. The results can be stored in a storage unit 529 and presented on a display 530. In some embodiments, an acoustical system can be configured to provide color flow images on a display in accordance with the acoustical measurements, though it is understood that color flow imaging is not required to practice some embodiments of the present application. Furthermore, some embodiments can utilize portions of a conventional color-flow image processing system (e.g., hardware and/or software) suitably modified to practice the techniques discussed in the present application.

The types of transducers that can be utilized with embodiments of the present invention include the range of acoustical transducers known to those skilled in the art. For example, multiple transducer elements can be arranged in a two-dimensional array, allowing for three-dimensional positioning of a sample volume for the received beam. For this particular system 500, the transducer 510 can act as both a signal transmitter and receiver, i.e., a transmitter of pulse energy and a receiver of echo signals. Other systems can utilize separate transducers for energy transmission and signal reception.

The pulse generator can be configured to activate a transducer to send one or more bursts of energy (e.g., in a high pulse repetition frequency mode), such as acoustic or ultrasonic pulses, to interact with flowing blood. When multiple bursts of energy are sent, optionally spaced in time, each burst can be configured to provide a set of backscattered signals corresponding with the burst. With respect to FIG. 5, the pulse generator 531 can be coupled to a beam estimator 532, which can be used to estimate beam profiles corresponding to the received signals to be processed. A beam estimator can be precalibrated to store, in some storage location 523, any number of potential beam profiles that can be used with an acoustic system to carry out various functionalities described in the present application.

The receiver can be configured to receive an echo signal corresponding to the backscattered signal from flowing blood interacting with a corresponding energy burst (e.g., ultrasonic pulse). The receiver can be coupled to a beamformer 534, which can operate in a conventional manner to convert the received echo signals into corresponding beams. For example, the beamformer 534 can utilize delay-and-sum beam forming followed by time gating to form a beam with a designated sample volume from the received echo signal. By utilizing the round-trip time for a pulse-echo signal to travel from and to a transducer(s) and the desired sample volume, the beamformer can utilize only the portion of the echo signal near the round-trip time such that the beam only interrogates the sample volume. Of course, the sample volume can also depend on the geometry of the transducer and the distance traveled by the signal. In some embodiments, the transducer and associated echo signal processors (e.g., receiver 533 and beam former 534) can be configured to form beams having cross sectional areas that are smaller than the cross sectional area of the orifice being investigated.

For the system 500, the beam former 534 is also coupled to an RF processor 535 to process the formed beam. Such processing can include a variety of signal modification techniques, including conventional steps typically utilized by those skilled in the art. In one embodiment, a RF processor can include a filter, which can be configured to remove portions of the echo signal relating to unwanted signal such as clutter, slow moving scatterers (e.g., a high-pass filter), and/or multiple echo signal interference. The formed beam is stored in a buffer 526 for later retrieval by the signal processor.

A signal processor 536 can be included in the system 500 for performing signal manipulations to produce one or more composite measures of blood flow characteristics as described herein (e.g., a spatial power distribution). As shown in FIG. 5, the signal processor 536 can receive echo signal data, which can be in the form of a beam that interrogates a sample volume. As well, the signal processor 536 can receive estimates of beam profiles from a beam estimator 532 and storage unit 523. Accordingly, a signal processor can be configured to utilize multiple pulse-echo beam profiles to estimate a composite beam profile. Such composite beam profiles can be utilized, e.g., with the echo signals, to create composite measures of blood flow characteristics such as a cross sectional area of the orifice that results in regurgitant blood flow.

Accordingly, several embodiments of the invention include signal processors, and other portions of an acoustic system, that can be configured to perform any number of the steps disclosed in the present application for forming composite measures of blood flow characteristics. Thus, a signal processor can be configured to utilize the methodologies described herein for estimating a cross sectional area of an orifice using equation (7), and the associated relationships discussed therewith. For example, a signal processor can be configured to calculate power measurements associated with the individual echo signals, as well as a composite power measurement. As well, other portions of a system 500, as depicted in FIG. 5, can also be configured to practice any portions of, or all of, the methodologies described herein. For example, the beam estimator 532, beamformer 534, and RF processor 535 can be configured along with the signal processor 536 to utilize a reference echo signal, a power measure and an associated reference echo signal profile in calculating a composite measure. The reference beam can be chosen in accordance with any of the techniques disclosed herein, such as choosing an echo signal formulated with cross sectional area in the orifice cross sectional area having maximum power, or averaging a number of signals to provide the reference signal or power. In another example, multiple sets of measurements can be combined (e.g., averaged) by the processor to provide the composite measure, where each set of measurements corresponds to interaction with blood flow at a unique time interval. Thus, combined values of overall power, reference power, and corresponding beam profiles, obtained by a system in accord with methods discussed herein, can be used to determine a composite measure such as a cross sectional area of the orifice.

The exemplary embodiment of FIG. 5 presents one particular arrangement of components for a system that can be used to obtain a composite measure of blood flow characteristics. Those skilled in the art will readily appreciate that various modifications can be made to the system. For example, the pulse generator 531, beam estimator 532, receiver 533, beamformer 534, RF processor 535, and signal processor 536 are depicted as separate processing elements. Such elements, however, can be combined into one physical processor, or combined in any number of ways to achieve the described functionality. Also, the processor can be embodied as a single physical device or a plurality of microprocessors that are integrated to perform the functions discussed herein. As well, the types of processor(s) that can be used to embody such elements include any processor capable of carrying out the desired functionality. Non-limiting examples of processors include programmable processors of a microcomputer, or processors with embedded memory or programming. Also, memory units 523, 526, 529 can be combined, kept separate, can be embedded within one or more physical processors, and can be embodied as one or more physical devices. All these variations, among others, are within the scope of the present application.

### EXAMPLES

The following examples are provided to illustrate some aspects of the present application. The examples, however, are not intended to limit the scope of any embodiment of the invention.

Aspects of the techniques described herein are exemplified by the following simulations that were implemented on a computer system running MATLAB software (The MathWorks, Natick, Mass.). In one simulation, a series of orifice cross sectional areas are estimated. For each estimated area, multiple beams are spaced apart at 0.5 times the Rayleigh criterion to estimate the area. Two-way focusing is assumed, and the one-way beam profiles are modeled as sinc functions.

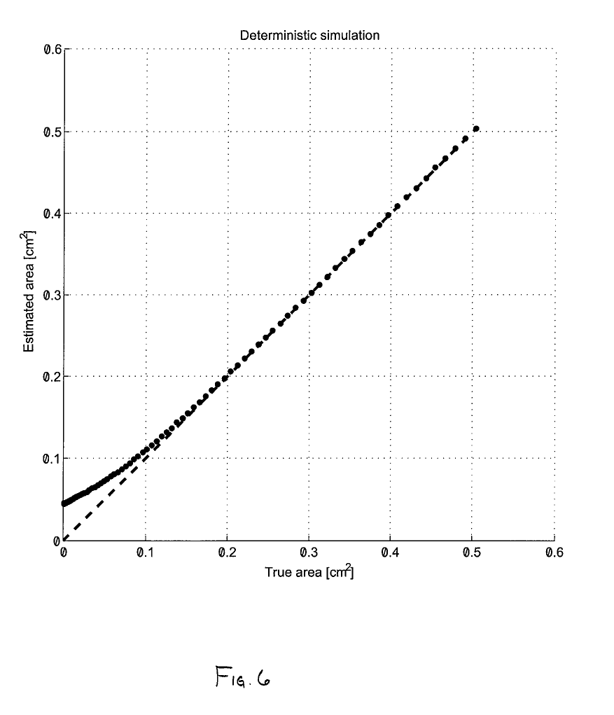

FIG. 6 provides a summary of the results of all the areas estimated by the simulation, where the vertical axis provides the orifice area values estimated using the techniques associated with equation (7), and the horizontal axis provides the “true” area values as programmed in the simulation. The dashed line indicates the line of identity, which would be the result if the estimates were perfectly accurate. The dotted line provides the comparison between the simulation estimates (y-axis value) and the programmed areas (x-axis value). In this example the technique is seen to provide good estimates of the cross sectional area of the orifice as long as the cross sectional area of the reference beam is smaller than the cross sectional area of the orifice. For orifices beneath this size the technique is seen to overestimate the cross sectional area of the orifice as expected. This is however not a problem if the examination is performed with settings similar to this simulation. A mitral- or aortic regurgitation with an orifice cross sectional area on the lower part of the scale, where the over-estimation occurs, pose no hazard to the patient according to the 2006 guidelines of the American College of Cardiology. These guidelines state that patients with mitral regurgitation orifices of cross sectional area smaller than 0.20 cm² are “mild”, orifices between 0.2 and 0.39 cm² are “moderate”, and orifices above 0.4 cm² are “severe”. So even with the overestimation of the smallest orifices the technique can easily distinguish between the levels of mitral regurgitation severities.

In this simulation the estimated power is simulated based on the amount of overlap between the beams and the orifice, with no stochastic modeling included. Thus the results are expected to be very good as long as the requirements of the technique are met, that is, the reference beam must be smaller than the orifice area, and the sum of beams constituting the measurement beam must be wide enough to cover the whole orifice.

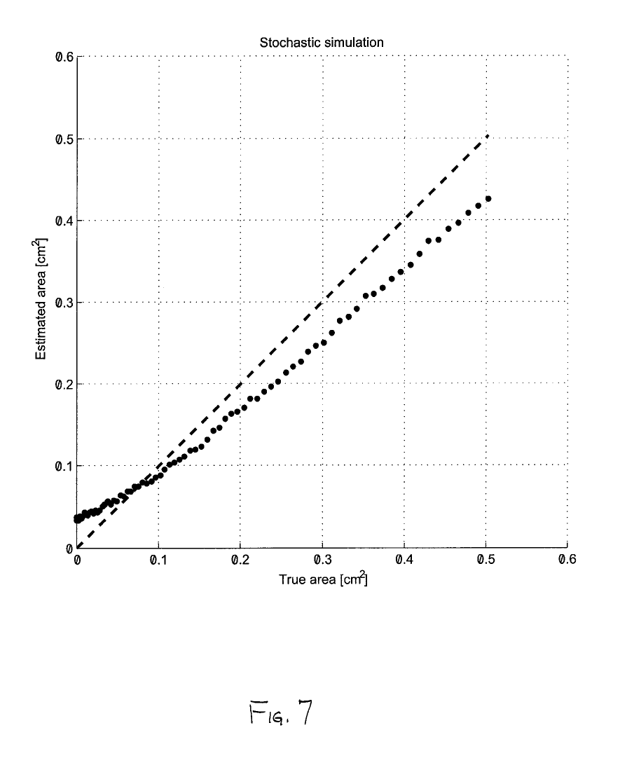

FIG. 7 presents the results of another simulation. Again a series of different cross sectional areas are estimated, but this time a simulation of blood flow is included. The simulation also includes Doppler-processing, determining the power estimates using the autocorrelation technique. The modeling of blood flow includes a stochastic element into the simulations, which causes the fluctuations of the estimates which can be seen from FIG. 7. The stochastic nature of the Doppler signal also leads to the bias of the estimate for the larger values of the orifice area. This under-estimation is expected as the reference beam is chosen amongst the beams with highest power, and for a larger orifice the probability of reaching a higher value for the reference beam increases. This can, however, easily be compensated for and should not be considered a problem with the technique. All other parameters are the same as used in the previous simulation. The descriptions for the axes and graphs are similar to those described for FIG. 6.

The common parameters of the two simulations above are shown in Table 1.

TABLE 1
Parameters for Flow Simulation

| Parameter | Value |
|---|---|
| Center Frequency fₒ | 2.5 MHz |
| Two-way azimuth | 2.5 |
| f-number | |
| Two-way elevation | 3.2 |
| f-number | |
| Wide beam | 15 × 15 |
| | beams |
| Number of Parallel | 4 |
| Beams | |
| Pulse Repetition | 22 kHz |
| Frequency | |
| Packet Size | 12 |
| Frame Rate | 33 |
| (Doppler only) | |

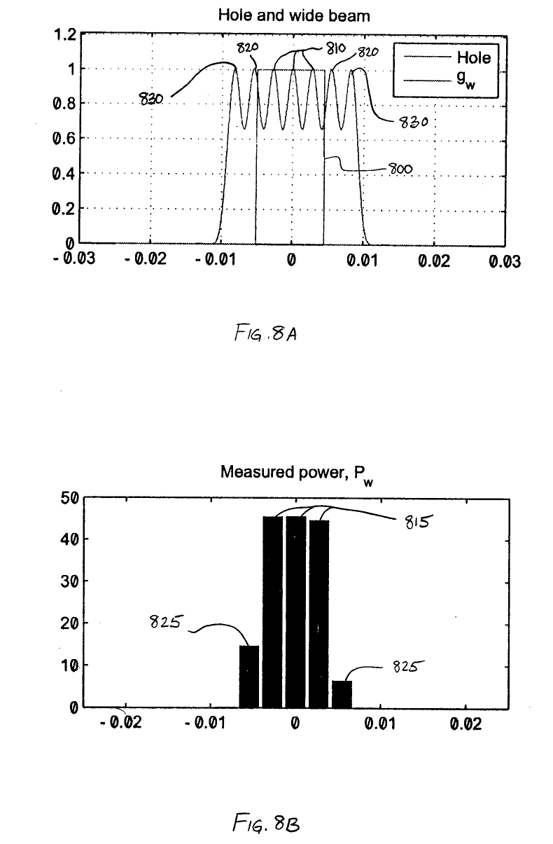

FIG. 8A provides a graph from a simplified 1-dimensional simulation showing a comparison between the location of the simulated orifice 800 and the one dimensional cross section of g_{w}(x,y,τ). FIG. 8B presents a graph of the power distribution as a function of position. In correspondence with FIG. 8A, the middle beams 810 each correspond with a bar 815 of FIG. 8B having a large power value. The beams corresponding with the edge of the orifice 820 have correspondingly lower power values 825. The beams completely outside the simulated orifice 830 show substantially no power value. Accordingly, the distribution shown in FIG. 8B also provides a composite measure of the geometry of the simulated orifice.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present invention. As well, one skilled in the art will appreciate further features and advantages of the invention based on the above-described embodiments. Accordingly, the invention is not to be limited by what has been particularly shown and described, except as indicated by the appended claims.

What is claimed is:

1. A method for estimating at least one characteristic of blood flow through an orifice of a blood vessel comprising:
acquiring a plurality of pulse-echo signals from a region of interest enveloping an opening area of the orifice, at least one of the pulse-echo signals having a sample area intersecting with the opening area of the orifice; and
assembling the plurality of pulse-echo signals to form a composite measure that provides an estimate of at least one blood flow characteristic through the orifice.

2. The method of claim 1, wherein the step of assembling includes generating a plurality of power signals each corresponding with at least one of the plurality of pulse-echo signals.

3. The method of claim 2, wherein the step of assembling further includes forming a composite power distribution.

4. The method of claim 1, wherein the composite measure provides an estimate of geometry of the orifice.

5. The method of claim 1, wherein the orifice is dynamic.

6. The method of claim 5, wherein the dynamic orifice is a heart valve.

7. The method of claim 1, wherein each of the pulse-echo signals is ultrasonic.

8. The method of claim 1, wherein the step of acquiring the plurality of pulse-echo signals includes using a high pulse repetition frequency technique.

9. The method of claim 1, wherein the step of acquiring the plurality of pulse-echo signals includes obtaining the plurality of pulse-echo signals from a vena contracta region of a jet blood flow through the orifice.

10. The method of claim 9, wherein the jet blood flow is from a regurgitant jet.

11. The method of claim 1, further comprising:
filtering the plurality of pulse-echo signals to suppress signal contributions from scatterers moving below a given velocity, before the step of assembling the plurality of pulse-echo signals.

12. The method of claim 1, further comprising:
estimating a plurality of pulse-echo beam profiles, each pulse-echo beam profile corresponding to one of the pulse-echo signals;
assembling the plurality of pulse-echo beam profiles to form a composite beam profile; and
estimating a cross sectional area of blood flow through the orifice using the composite beam profile.

13. The method of claim 12, wherein the step of estimating the cross sectional area of blood flow comprises:
estimating a total power of the plurality of pulse-echo signals;
identifying a reference pulse-echo signal from the plurality of pulse-echo signals, and a corresponding reference signal power;
identifying a reference beam profile, corresponding to the reference pulse-echo signal, from the estimated plurality of pulse-echo beam profiles; and
calculating the cross sectional area of blood flow from the estimated total power of the plurality of pulse-echo signals, the reference signal power, the reference beam profile, and the composite beam profile.

14. The method of claim 13, wherein estimating the total power includes estimating multiple set-total power values from corresponding sets of pulse-echo signals, each set of pulse-echo signals corresponding with backscattering during a unique time interval, and calculating the total power by combining the multiple set-total power values.

15. The method of claim 14, wherein identifying the reference signal power includes combining multiple signal power values, each signal power value corresponding with one of the plurality of pulse-echo signals that has cross sectional area located within the opening area of the orifice.

16. The method of claim 15, wherein identifying a reference signal power includes identifying spatially corresponding reference pulse-echo signals, and combining time-varying power values, each time varying power value corresponding with one of the spatially corresponding reference pulse-echo signals.

17. The method of claim 13, wherein the identified reference pulse-echo signal corresponds with one of the plurality of pulse-echo signals having highest power.

18. The method of claim 13, further comprising:
utilizing the plurality of pulse-echo signals to estimate an average velocity of blood flow through the orifice; and
estimating a blood flow rate through the orifice using the average velocity of blood flow and the calculated cross sectional area.

19. The method of claim 18, wherein the step of utilizing the plurality of pulse-echo signals includes determining a blood flow velocity corresponding with each of the pulse-echo signals.

20. The method of claim 1, wherein the composite measure is obtained without performing spectral analysis on signals derived from the pulse-echo signals.

21. An acoustical system for interrogating blood flow through an orifice of a blood vessel, comprising:
a pulse generator for transmitting bursts of energy towards an opening area of the orifice;
at least one transducer for receiving multiple echo signals, each echo signal corresponding to a backscattered signal from at least one of the transmitted bursts of energy; and
a signal processor coupled to the at least one transducer, the signal processor configured to convert the multiple echo signals into a composite measure of at least one blood flow characteristic through the orifice.

22. The system of claim 21, wherein the at least one transducer for receiving the multiple echo signals is also coupled to the pulse generator, and configured to transmit the bursts of energy.

23. The system of claim 21, wherein the at least one transducer is adapted to form each echo signal into a beam having a sample area intersecting with blood flow through the orifice.

24. The system of claim 21, wherein the pulse generator is configured to generate pulsed ultrasonic energy.

25. The system of claim 21, wherein the pulse generator is configured to provide the bursts of energy in a high pulse repetition frequency mode.

26. The system of claim 21, further comprising:
a high-pass filter coupled to the signal processor, the high-pass filter configured to remove portions of signals corresponding to slowly moving scatterers from the multiple echo signals.

27. The system of claim 21, wherein the pulse generator is configured to produce a plurality of sets of transmitted energy bursts, each transmitted energy burst of a set adapted to backscatter in the opening area during a unique time interval.

28. The system of claim 27, wherein the signal processor is configured to calculate a combined-echo-signal power from a plurality of sets of multiple echo signals, each set of echo signals corresponding to a particular set of transmitted energy bursts, each set of echo signals being converted to a set-total power value, the set-total power values being combined to form the combined-echo-signal power.

29. The system of claim 21, wherein the signal processor is configured to associate an individual echo beam profile with each echo signal, and to estimate a composite beam profile from a combination of the individual echo beam profiles.

30. The system of claim 29, wherein the signal processor is configured to convert the multiple echo signals into a composite spatial power distribution, the signal processor further configured to estimate a cross sectional area of a blood flow jet emitted from the orifice, the estimate of the cross sectional area being based upon a composite power value derived from the composite spatial power distribution, and the composite beam profile.

31. The system of claim 30, wherein the signal processor is configured to estimate the cross sectional area of the blood flow jet by choosing a reference echo signal from the multiple echo signals, the at least one transducer configured to modify the reference echo signal to correspond to a sample area intersecting the blood flow jet.

32. The system of claim 31, wherein the reference echo signal corresponds with one of the multiple echo signals having maximum power.

33. The system of claim 31, wherein the reference echo signal corresponds to an average signal from a plurality of the multiple echo signals each having a cross sectional area within the opening area.

34. The system of claim 21, wherein the system is configured to produce color flow images.

35. The system of claim 21, wherein the signal processor is configured to form the composite spatial power distribution without utilizing spectral analysis on received echo signals.
